(19) United States
(12) Patent Application Publication
(10) Pub. No.: US 2024/0066335 A1
(43) Pub. Date: Feb. 29, 2024

(54) STORAGE CASE FOR A PORTABLE DEVICE

(71) Applicant: Dyson Technology Limited, Wiltshire (GB)

(72) Inventor: Nikian Naji AGHABABAIE, London (GB)

(73) Assignee: Dyson Technology Limited, Wiltshire (GB)

(21) Appl. No.: 18/267,745

(22) PCT Filed: Oct. 7, 2021
(86) PCT No.: PCT/GB2021/052598

§ 371 Date: Jun. 15, 2023

(30) Foreign Application Priority Data
Dec. 18, 2020 (GB) 2020105.9

(51) Int. Cl. A62B 25/00; A45C 11/00; A45C 13/02; A45C 15/06; A61L 2/10; A61L 2/26; A62B 18/08; A62B 23/00; B01D 46/00; H02J 7/00

(52) U.S. Cl. CPC A62B 25/00; A45C 11/00; A45C 13/02; A45C 15/06; A61L 2/10; A61L 2/26; A62B 18/08; A62B 23/00; B01D 46/0028; B01D 46/0091; H02J 7/0045; A61L 2202/11; A61L 2202/122; A61L 2202/14; A61L 2202/16; A62B 18/006

(57) ABSTRACT

A storage case is provided for a portable device, the storage case including at least one docking bay and at least one light source. The docking bay is shaped for holding at least a part of the portable device in a predetermined position. The light source is configured for emitting light in a violet portion of the visual spectrum, and arranged in such a way as to illuminate a specific portion of the portable device when the at least one part of the portable device is held in the predetermined position.

### TECHNICAL FIELD

The present invention relates to a storage case for a portable device.

### BACKGROUND

Over recent years, public interest in air quality in both indoor and outdoor environments has increased. As a result, many people have been buying air purifying devices for their homes and offices. Air purifiers generally use a compressor to take in air from the direct environment of the air purifier, one or more filters to remove unwelcome particles and other contaminants, and one or more nozzles from which to expel the filtered outgoing air. Depending on the application, the air may not just be filtered, but also cooled, heated, moisturised, dried, or otherwise treated, while it flows between the air inlet and an outlet of the air purifier.

In the international patent application published as WO 2020/021231 A1, a head wearable air purifier is disclosed. The head wearable air purifier, which also comprises speaker assemblies to double as a pair of headphones, comprises an impeller for drawing air in through an air inlet and a filter assembly and for expelling the air from an air outlet downstream from the filter assembly. An arcuate nozzle is connected to the air outlet to guide the expelled air towards an array of nozzle outlets that, in use, are arranged in front of a user's mouth to ensure that the user breathes in clean and filtered air.

A problem with these wearable air purifiers is that the air drawn in at the air inlets, is obtained from an environment that may contain all kinds of contamination. Larger dirt and dust particles may be filtered out, but smaller contaminants such as bacteria and other microbes are drawn in too. Such microbial contamination may then be directly blown towards the user or may gather and grow around the nozzles, in the filters, or elsewhere inside the air ducts of the air purifier. When users touch the nozzle with their hands, mouth or other parts of their face, contamination may also occur due to such direct contact. Use, or even just handling, of the same wearable air purifier by different persons will further increase the health and safety risks associated with such contamination.

Similar contamination risks may also be present in other portable devices that are carried around in public and are used and touched in places where the user may come into close contact with many different other people. Cleaning of a wearable air purifier or other portable devices will often be done with a dry or wet cloth, which will likely lead to more microbial contamination, rather than less. Therefore, there is a need for ways to better protect the users of such wearable air purifiers against contact with unwanted microbes while cleaning their homes and offices.

It is an aim of the present invention to address one or more disadvantages associated with the prior art.

### SUMMARY OF THE INVENTION

According to an aspect of the invention there is provided a storage case for a portable device, the storage case comprising at least one docking bay and at least one light source. The docking bay is shaped for holding at least a part of the portable device in a predetermined position. The light source is configured for emitting light in a violet portion of the visual spectrum, and arranged in such a way as to illuminate a specific portion of the portable device when the at least one part of the portable device is held in the predetermined position.

The violet portion of the visual spectrum is typically defined as spanning the range of about 380 to 450 nm. The light used may thus, e.g., have a wavelength of about 405 nm. Light of these wavelengths is known to be very effective in killing any microbes that may have accumulated on the illuminated surfaces. Although such light is known to be used in light fixtures used for cleaning rooms in hospitals and in standalone curing lights used for 3D printer resin and nail polish, it has so far not been used in storage cases for portable devices. The use of violet visible light for this particular implementation brings a number of advantages that are not found in UV or near UV light. For example, the low energy visible light does not damage the material of the surfaces it illuminates. This is especially advantageous because many portable devices and their storage cases are at least partially made of plastics that are easily damaged by UV light. Another important advantage of the violet visible light is that no direct line of sight between the light source and the surface or part to be cleaned is needed. Indirect irradiation of the violet visible light helps to get rid of the microbial contamination too.

Integrating the illumination and decontamination means into the storage case brings the advantage that even though the portable device may not have any decontamination means of its own, the decontamination can start as soon the user puts away the portable device and can continue up to the point when the user removes it from its storage case to start using it again. This allows the portable device to be decontaminated while away from home or other places where the user may be able to properly clean the device. Further, when the decontamination program has been completed, typically after between 30 to 120 minutes of illumination with violet light, a closed storage case may protect the portable device against recontamination.

It is to be noted that emitting light in a violet portion of the visual spectrum as part of a decontamination process means that the emitted light contains a significant portion of light in that part of the electromagnetic spectrum and that the intensity of that significant portion is sufficient to have a useful anti-microbial and decontaminating effect. The emitted light does not need to be exclusively in the violet portion of the visual spectrum. As long as there is a sufficient intensity of light in that portion of the spectrum, and preferably at or around the 405 nm wavelength, for achieving a decontaminating effect, light of other parts of the electromagnetic spectrum may be emitted too. Further it is noted that, as part of the decontamination process, the intensity of the emitted light may vary over time. Such variations may be gradual and continuous or in the form of a pattern of light pulses. If pulsed light is used, the frequency, duration and intensity of the pulses may either be constant or varying.

In the context of the current invention, the term ‘portable’ is to be interpreted as being sufficiently compact and lightweight to be carried around by its user while walking. Because of its portability, the portable device is preferably cordless and battery powered, but corded devices may be portable too. In a special embodiment, the portable device may be wearable, e.g. on the user's wrist or head. Wearable devices can be used while keeping the user's hands free for other tasks.

Optionally, the storage case further comprises a power source, coupled to the at least one light source for providing electrical power thereto. The power may, e.g., comprise a battery. The battery may be rechargeable. When the storage case comprises its own power source, the light source does not need to be powered by a battery of the portable device and the decontamination does not reduce the amount of time that the portable device can be used without charging or battery replacement.

In a preferred embodiment, the storage case further comprises a device charger, coupled to the power source and configured to charge a battery of the portable device when the part of the portable device is held in the predetermined position. Most portable devices are designed to be compact and low weight. To make this possible, often a compromise on battery capacity needs to be made. With the possibility to charge the portable device using a battery of the storage case, the total battery capacity available for the use of the portable device is increased without adding weight or size to the portable device itself.

In an alternative embodiment, the storage case may comprise a power receiver, coupled to the at least one light source and configured to receive power from a power source of the portable device when the part of the portable device is held in the predetermined position. This brings the advantage that the storage case can be kept low weight and relatively low cost. This configuration may be especially useful when used for portable devices for which battery lifetime is not a major issue.

In an embodiment of the invention, the storage case further comprises a case controller, operatively coupled to the at least one light source and operative to control the at least one light source to illuminate the at least one part of the portable device. The controller may be configured to run a special decontamination program. The decontamination program controls which light source is turned on when and for how long. The decontamination program may be predetermined and may, e.g., start when the portable device is stored into its predetermined position, when the storage case is closed, when the storage case is connected to an external power source and/or when the user starts the decontamination program. Alternatively, the decontamination program is controlled by a controller of the portable device, which may be coupled to the at least one light source when placing the portable device in its predetermined position inside the storage case.

The storage case may further comprise a light guide configured to guide the light emitted by the at least one light source to the specific portion of the portable device. Although decontamination by light in the violet portion of the visual spectrum is possible with indirect irradiation, higher light intensities can lead to better decontamination results. A light guide in the storage case may therefore be configured to guide the decontaminating light to exactly those parts of the portable device that are either the most susceptible to contamination or pose the highest risk of passing on contamination to the user. A similar effect may be achieved by providing a plurality of light sources at specifically selected positions, however this may not be possible or easy to realise in all important locations.

In preferred embodiments, the portable device is a personal air purifier, e.g. one similar to the personal air purifier described in WO 2020/021231 A1. The personal air purifier may comprise a filter assembly with an air inlet and an air filter, and a visor with an air outlet. The at least one docking bay of the storage case may comprise a first docking bay, shaped for holding the filter assembly in a predetermined position, and a second docking bay, shaped for holding the visor in a predetermined position. The at least one light source comprises a filter assembly light source, arranged in such a way as to illuminate the filter assembly when held in its predetermined position, and a visor light source, arranged in such a way as to illuminate the visor when held in its predetermined position. When in the storage case, the visor may be detached from the filter assembly.

The portable device may further comprise a light guide configured to guide the light emitted by the at least one light source to the specific portion of the portable device. Even though the light source may be part of the storage case, the light guide may be part of the product design of the portable device.

### BRIEF DESCRIPTION OF THE DRAWINGS

Embodiments of the invention will now be described by way of example with reference to the accompanying drawings, in which:

FIG. 1 shows a portable device in the form of a head wearable air purifier.

FIG. 2 shows a top view of an embodiment of a storage case according to the invention, wherein a wearable air purifier is stored.

FIG. 3 shows a perspective view of the storage case and wearable air purifier of FIG. 2.

### DETAILED DESCRIPTION

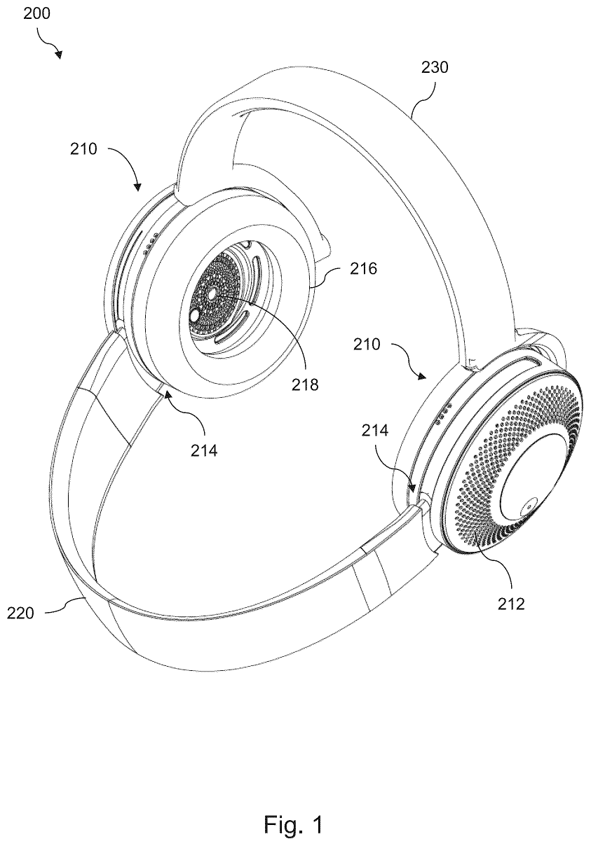

FIG. 1 shows a portable device in the form of a wearable air purifier 200. The head wearable air purifier 200 comprises a pair of generally cylindrical speaker assemblies 210 connected by an arcuate headband 230, and a nozzle or visor 220 that extends between and is connected at opposite ends to both speaker assemblies 210. Each of the pair of speaker assemblies 210 comprises an air inlet 212 and an air outlet or discharge port 214, a speaker or driver unit within the housing, and an earpad 216 arranged to enclose the speaker 218 and to encompass or press against an ear of a user. Inside its housing, each of the pair of speaker assemblies 210 further comprises a filter assembly, an impeller for creating an airflow through the filter assembly and a motor arranged to drive the impeller. The air outlet or discharge port 214 is downstream (i.e. relative to the airflow generated by the impeller) from the filter assembly and is arranged to emit the filtered/purified airflow from the speaker assembly 210. In the illustrated embodiment, the air outlet or discharge port 214 of each speaker assembly 210 is provided in a side of the speaker assembly 210, with the air outlet or discharge port 214 of both speaker assemblies 210 being connected to the nozzle or visor 220. In use, the speaker assemblies 210 are positioned on the user's ears, the headband 230 on top of the user's head and the visor 220 in front of the user's mouth. Air outlets provided at the mouth facing side of the visor 220 blow the filtered and purified air towards and into the user's mouth, thereby allowing the user to breath in air that is considerably cleaner than the ambient air of her direct environment.

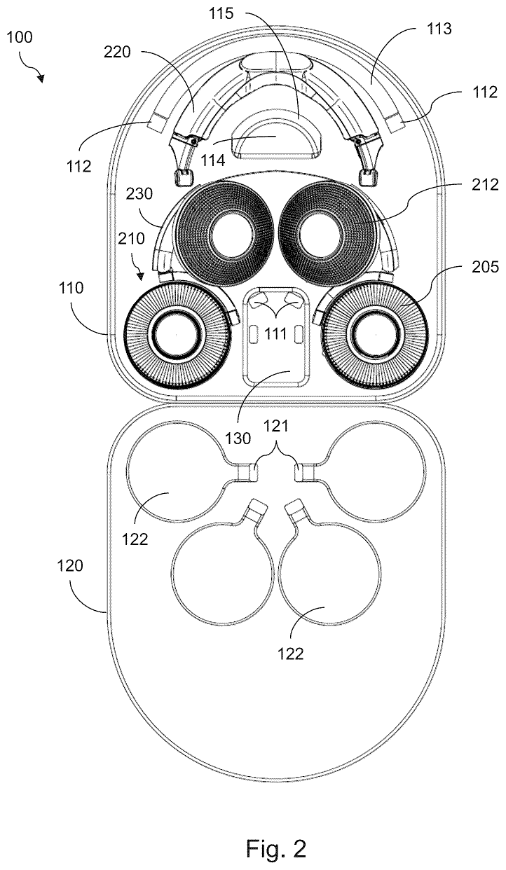

FIG. 2 shows a top view of an embodiment of a storage case 100 according to the invention, wherein a wearable air purifier 200 is stored. The storage case 100 comprises a bottom portion 110 and a cover 120. The storage case 100 is designed such that the wearable air purifier 200 can only be stored in a specific predetermined position and orientation. In this preferred embodiment, the wearable air purifier 200 is partly disassembled in order to make it easier to thoroughly clean all critical parts of the device 200. The air inlet covers 212 of the speaker assemblies 210 are stored in their own dedicated storage locations, separate from the housing and other parts of the speaker assemblies 210. By the removal of the air inlet covers 212, the outer surface of the filter assemblies 205 are revealed and made directly accessible for the decontaminating light that is provided by the storage case. The visor 220 is disconnected from the air outlets 214 of the speaker assemblies 210 and stored in its own separate docking bay. Preferably, the bottom surface and/or the walls of the bottom portion 110 comprise some 3D features designed to keep the disassembled parts of the wearable air purifier firmly in place when stored.

A control box 130, here provided in the bottom portion 110 of the storage case may comprise a battery and a controller for controlling the decontamination process. The battery may be rechargeable. In addition to controlling the decontamination process, the controller may also perform other control tasks, such as controlling the charging of a battery of the wearable air purifier 200. In the same control box 100, light sources, here in the form of LEDs 111, are provided that are configured for emitting light in the violet part of the visual spectrum, e.g. with a wavelength of about 405 nm. Additional light sources 112, 114 are provided at different locations in the bottom portion 110 of the storage case 100. These additional light sources 112, 114 are preferably electrically connected to the controller and battery inside the control box 130 too. Optionally, further light sources may be provided in the cover 120 of the storage case 100. Such light sources may be coupled to the battery and/or controller in the control box 130 via a wired connection passing through a hinge of the storage case 100, via an electrical contact that is made when the storage case 100 is closed, or via a wireless connection. Alternatively, a separate power source and/or controller is provided in the cover 120 itself.

Light guides 113, 115 provided in the bottom portion 110 of the storage case 100 may be coupled to the light sources 112, 114 for guiding the light to those parts of the wearable air purifier 200 that need to be decontaminated. The light guides 113, 115, e.g., ensure that the complete inner and outer surfaces of the visor 220 are thoroughly cleaned. In addition thereto, the light guides 113, 115 form the docking bay that can hold the visor 220 and keep it in place when stored inside the storage case 100.

Additional light guides 121, 122 may be provided in the cover 120 of the storage case 100. The light guides 121, 122 in the cover 120 have a connection portion 121 and cleaning portion 122. The connection portions 121 are designed such that when the cover is closed, they are in direct contact with or close proximity to the LEDs 111 in the control box 130 of the bottom portion 110. When the cover 120 is closed and the LEDs 111 are on, the connection portions 121 direct the violet light to the cleaning portions 122 of the light guides. These cleaning portions 122 provide a large surface area that roughly matches the shape and size of the air inlet covers 212 and the filter assemblies 205 they need to decontaminate. One cleaning portion 122 is provided for each inlet cover 212 and for each filter assembly 205. Optionally, a similar light guiding surface may be provided in the cover 120 of the storage case 100 to decontaminate the top surface of the visor 220. Such additional light guiding surface may receive the violet light from the light guides 113, 115 in the bottom portion 110 that are arranged along the inner and outer surfaces of the visor 220.

The controller in the control box 130 may be configured to run a special decontamination program which controls which light source 111, 112 is turned on when and for how long. The decontamination program may be predetermined and may, e.g., start when the air purifier 200 or one or more parts of the air purifier 200 are stored into their predetermined positions. Alternatively, the decontamination starts when the storage case 100 is closed, when the storage case 100 is connected to an external power source or when the user starts the decontamination program.

Alternatively, the decontamination program is controlled by a controller in one of the speaker assemblies 210 and powered by a battery that is also part of the air purifier 200. To make this possible, the speaker assembly 215 may be electronically coupled to light sources 111, 112 of the storage case by placing the air purifier parts in their predetermined positions inside the storage case 100.

The decontamination program may have a fixed duration, or a duration that depends on, e.g., whether the light sources 111, 112 are powered by a battery or through a connection to a power socket. The light sources 111, 112 may emit light continuously or in a pulsed manner. The light intensity may be constant or variable. The duration of the decontamination program may depend on the selected light intensity. When, e.g., a quick decontamination program is desired, the light sources 111, 112 may emit light at a higher intensity but for a shorter period of time. The exact lighting pattern used for the decontamination program may be pre-programmed and/or adjustable. The user may be able to start, pause, stop, or adjust the decontamination programs via buttons or a user interface on the storage case 100 or on one of the speaker assemblies 210. Alternatively, the user may control the process via a smartphone app that communicates with the controller in one of the speaker assemblies 210 and/or the control box 130 in the storage case 100. If the storage case 100 has a charging function in addition to the decontamination functions, the user may be allowed to only charge, only decontaminate or two do both simultaneously.

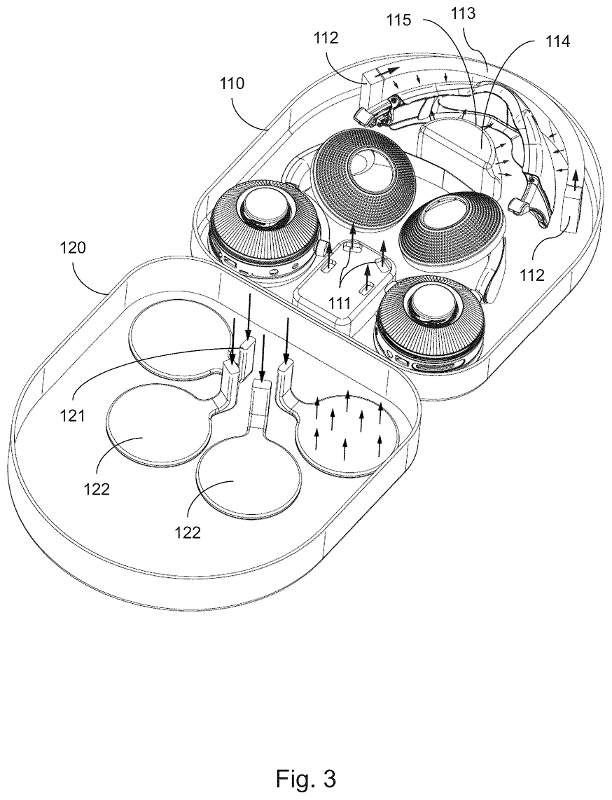

FIG. 3 shows a perspective view of the storage case 100 and wearable air purifier 200 of FIG. 2. In addition to what was already shown in FIG. 2, FIG. 3 shows indicates with arrows how the violet light is emitted by the various light sources 111, 112, 114 guided through the light guides 121, 122, 113, 115 and from the light guides 121, 122, 113, 115 to the various parts of the wearable air purifier 200.

The invention has been described above in relation to a number of different embodiments. It is to be noted that storage case according to the invention, although particularly useful in combination with portable and wearable air purifiers, is equally useful to and compatible with other types of portable devices. Further, features used in and described with reference to specific embodiments are combinable with other embodiments. The scope of the invention is only limited by the following claims.

1. A storage case for a portable device, the storage case comprising:
at least one docking bay, shaped for holding at least a part of the portable device in a predetermined position, and
at least one light source for emitting light in a violet portion of the visual spectrum, the at least one light source being arranged in such a way as to illuminate a specific portion of the portable device when the at least one part of the portable device is held in the predetermined position.

2. The storage case according to claim 1, wherein the at least one light source is configured for emitting light with a wavelength of about 405 nm.

3. The storage case according to claim 1, further comprising a power source, coupled to the at least one light source for providing electrical power thereto.

4. The storage case according to claim 3, further comprising a device charger, coupled to the power source and configured to charge a battery of the portable device when the part of the portable device is held in the predetermined position.

5. The storage case according to claim 3, wherein the power source comprises a rechargeable battery.

6. The storage case according to claim 1, further comprising a power receiver, coupled to the at least one light source and configured to receive power from a power source of the portable device when the part of the portable device is held in the predetermined position.

7. The storage case according to claim 1, further comprising a case controller, operatively coupled to the at least one light source and operative to control the at least one light source to illuminate the at least one part of the portable device.

8. The storage case according to claim 1, further comprising a light guide configured to guide the light emitted by the at least one light source to the specific portion of the portable device.

9. The storage case according to claim 1, further comprising the portable device, the part of the portable device being held in the predetermined position.

10. The storage case according to claim 9, wherein the portable device is a personal air purifier.

11. The storage case according to claim 10, wherein the personal air purifier comprises
a filter assembly with an air inlet and an air filter, and
a visor with an air outlet,
the at least one docking bay comprising a first docking bay, shaped for holding the filter assembly in a predetermined position, and a second docking bay, shaped for holding the visor in a predetermined position,
the at least one light source comprising a filter assembly light source, arranged in such a way as to illuminate the filter assembly when held in its predetermined position, and a visor light source, arranged in such a way as to illuminate the visor when held in its predetermined position.

12. The storage case according to claim 11, wherein the visor is detached from the filter assembly.

13. The storage case according to claim 9, wherein the portable device further comprises a device controller, the storage case and the portable device each comprising a coupling unit for operatively coupling the device controller to the at least one light source when the part of the portable device is held in the predetermined position, the device controller being operative to control the at least one light source to illuminate the at least one part of the portable device.

14. The storage case according to claim 9, wherein the portable device further comprises a light guide configured to guide the light emitted by the at least one light source to the specific portion of the portable device.
